SEQUENCE LISTING (44 sequences)

SEQ ID NO: 1 - DNA, 2840 bp, human
atgacaagaaggacccagcctccgagcggccacaccctgtgtgtctctctgtcctgccagcactgagggctcatccctctgcagagcgcggggtcaccgggaggagacgccatgacgcccgccctcacagccctgctctgccttgggctgagtctgggccccaggacccgcgtgcaggcagggcccttccccaaacccaccctctgggctgagccaggctctgtgatcagctgggggagccccgtgaccatctggtgtcaggggagccaggaggcccaggagtaccgactgcataaagagggaagcccagagcccttggacagaaataacccactggaacccaagaacaaggccagattctccatcccatccatgacagagcaccatgcagggagataccgctgccactattacagctctgcaggctggtcagagcccagcgaccccctggagatggtgatgacaggagcctacagcaaacccaccctctcagccctgcccagccctgtggtggcctcaggggggaatatgaccctccgatgtggctcacagaagggatatcaccattttgttctgatgaaggaaggagaacaccagctcccccggaccctggactcacagcagctccacagtcgggggttccaggccctgttccctgtgggccccgtgacccccagccacaggtggaggttcacatgctattactattatacaaacaccccctgggtgtggtcccaccccagtgaccccctggagattctgccctcaggcgtgtctaggaagccctccctcctgaccctgcagggccctgtcctggcccctgggcagagcctgaccctccagtgtggctctgatgtcggctacaacagatttgttctgtataaggagggggaacgtgacttcctccagcgccctggccagcagccccaggctgggctctcccaggccaacttcaccctgggccctgtgagcccctccaatgggggccagtacaggtgctacggtgcacacaacctctcctccgagtggtcggcccccagcgaccccctgaacatcctgatggcaggacagatctatgacaccgtctccctgtcagcacagccgggccccacagtggcctcaggagagaacgtgaccctgctgtgtcagtcatggtggcagtttgacactttccttctgaccaaagaaggggcagcccatcccccactgcgtctgagatcaatgtacggagctcataagtaccaggctgaattccccatgagtcctgtgacctcagcccacgcggggacctacaggtgctacggctcatacagctccaacccccacctgctgtctcaccccagtgagcccctggagctcgtggtctcaggacactctggaggctccagcctcccacccacagggccgccctccacacctggtctgggaagatacctggaggttttgattggggtctcggtggccttcgtcctgctgctcttcctcctcctcttcctcctcctccgacgtcagcgtcacagcaaacacaggacatctgaccagagaaagactgatttccagcgtcctgcaggggctgcggagacagagcccaaggacaggggcctgctgaggaggtccagcccagctgctgacgtccaggaagaaaacctctatgctgccgtgaaggacacacagtctgaggacagggtggagctggacagtcagagcccacacgatgaagacccccaggcagtgacgtatgccccggtgaaacactccagtcctaggagagaaatggcctctcctccctcctcactgtctggggaattcctggacacaaaggacagacaggtggaagaggacaggcagatggacactgaggctgctgcatctgaagcctcccaggatgtgacctacgcccagctgcacagcttgacccttagacggaaggcaactgagcctcctccatcccaggaaggggaacctccagctgagcccagcatctacgccactctggccatccactagcccggggggtacgcagaccccacactcagcagaaggagactcaggactgctgaaggcacgggagctgcccccagtggacaccagtgaaccccagtcagcctggacccctaacacagaccatgaggagacgctgggaacttgtgggactcacctgactcaaagatgactaatatcgtcccattttggaaataaagcaacagacttctcaacaatcaatgagttaataacaaaaaaacaaaaaacaaaaacagacgtaaaggccgggtgtggtactcaggaggctgagtggggaggattccttgaacacaagaagttaaggctgctgaggctgcagtgagctatgactgtgccactgcactccagcctgtgtgacagagcgagaccttgtctctaaaaaaaaaaacagtgaatgttttaaactgaatgataatgtaaatattatacatcgaacttatgacatgggaaaattaagaagcataaataggccgggcgcggtggctcacgcctataatctcagcactttgggaggctgatgcgggcggatcatgaggtcaggagatcgagaccatcctggctaacacggtgaaaccccgtctctactaaaaatacaaaaaaattagccgggcgtggtggcgagtgcctatagtcccagctactcaggaggctgaggcaggagaatggcatgagcccgggaggcagagcttgcagtgagctgagatcgcaccactgcactccagcctgggcgacagagtgagattccgtctcgaaaaaaaaaaaaaaagaaagaaaaaaaataaaaaagaagcataaccaggaaaaaaaaaaaaaaaaaa

SEQ ID NO: 2 - DNA, 538 bp, human
agaaagactgatttccagcgtcctgcaggggctgcggagacagagcccaaggacaggggcctgctgaggaggtccagcccagctgctgacgtccaggaagaaaacctctagcccacacgatgaagacccccaggcagtgacgtatgccccggtgaaacactccagtcctaggagagaaatggcctctcctccctcctcactgtctggggaattcctggacacaaaggacagacaggtggaagaggacaggcagatggacactgaggctgctgcatctgaagcctcccaggatgtgacctacgcccagctgcacagcttgacccttagacggaaggcaactgagcctcctccatcccaggaaggggaacctccagctgagcccagcatctacgccactctggccatccactagcccggggggtacgcagaccccacactcagcagaaggagactcaggactgctgaaggcacgggagctgcccccagtggacaccagtgaaccccagtcagcctggacccctaacacagaccatgagga

SEQ ID NO: 3 - DNA, 1807 bp, human
gcggccgcgtctcctccctcggcgttgtccgcggcgcgagccacagcgcgcggggcgagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgacatgggggaccgggagcagctgctgcagcgggcgcggctggccgagcaggcggagcgctacgacgacatggcctccgctatgaaggcggtgacagagctgaatgaacctctctccaatgaagatcgaaatctcctctctgtggcctacaagaatgtggttggtgccaggcgatcttcctggagggtcattagcagcattgagcagaaaaccatggctgatggaaacgaaaagaaattggagaaagttaaagcttaccgggagaagattgagaaggagctggagacagtttgcaatgatgtcctgtctctgcttgacaagttcctgatcaagaactgcaatgatttccagtatgagagcaaggtgttttacctgaaaatgaagggtgattactaccgctacttagcagaggtcgcttctggggagaagaaaaacagtgtggtcgaagcttctgaagctgcctacaaggaagcctttgaaatcagcaaagagcagatgcaacccacgcatcccatccggctgggcctggccctcaacttctccgtgttctactatgagatccagaatgcacctgagcaagcctgcctcttagccaaacaagccttcgatgatgccatagctgagctggacacactaaacgaggattcctataaggactccacgctgatcatgcagttgctgcgagacaacctcaccctctggacgagcgaccagcaggatgaagaagcaggagaaggcaactgaagatccttcaggtcccctggcccttccttcacccaccacccccatcatcaccgattcttccttgccacaatcactaaatatctagtgctaaacctatctgtattggcagcacagctactcagatctgcactcctgtctcttgggaagcagtttcagataaatcatgggcattgctggactgatggttgctttgagcccacaggagctccctttttgaattgtgtggagaagtgtgttctgatgaggcattttactatgcctgttgatctatgggaaatctaggcgaaagtaatggggaagattagaaagaattagccaaccaggctacagttgatatttaaaagatccatttaaaacaagctgatagtgtttcgttaagcagtacatcttgtgcatgcaaaaatgaattcacccctcccacctctttcttcaattaatggaaaactgttaagggaagctgatacagagagacaacttgctcctttccatcagctttataataaactgtttaacgtgaggtttcagtagctccttggttttgcctctttaaattatgacgtgcacaaaccttcttttcaatgcaatgcatctgaaagttttgatacttgtaacttttttttttttttggttgcaattgtttaagaatcatggatttattttttgtaactctttggctattgtccttgtgtatcctgacagcgccatgtgtgtcagcccatgtcaatcaagatgggtgattatgaaatgccagacttctaaaataaatgttttggaattcaatgggtaaataaatgctgctttggggatattaaaaaaaaaaaaaaa

SEQ ID NO: 4 - DNA, 547 bp, human
agctgagctggacacactaaacgaggattcctataaggactccacgctgatcatgcagttgctgcgagacaacctcaccctctggacgagcgaccagcaggatgaagaagcaggagaaggcaactgaagatccttcagatcccctggcccttccttcacccaccacccccatcatcaccgattcttccttgccacaatcactaaatatctagtgctaaacctatctgtattggcagcacagctactcagatctgcactcctgtctcttgggaagcagtttcagataaatcatgggcattgctggactgatggttgctttgagcccacaggagctccctttttgaattgtgtggagaagtgtgttctgatgaggcattttactatgcctgttgatctatgggaaatctaggcgaaagtaatggggaagattagaaagaattagccaaccaggctacagttgatatttaaaagatccatttaaaacaagctgatagtgtttcgttaagcagtacatcttgtgcatgcaaaaatgaattcacccctccca

SEQ ID NO: 5 - DNA, 2439 bp, human
tgcagagcgcatgctctggggcagttcgcggcccggcggggagcgccggagttccttgtggccgacgtgcaccaaggacaggaagatgtcacccaagcgcatagctaaaagaaggtcccccccagcagatgccatccccaaaagcaagaaggtgaaggtctcacacaggtcccacagcacagaacccggcttggtgctgacactaggccagggcgacgtgggccagctggggctgggtgagaatgtgatggagaggaagaagccggccctggtatccattccggaggatgttgtgcaggctgaggctgggggcatgcacaccgtgtgtctaagcaaaagtggccaggtctattccttcggctgcaatgatgagggtgccctgggaagggacacatcagtggagggctcggagatggtccctgggaaagtggagctgcaagagaaggtggtacaggtgtcagcaggagacagtcacacagcagccctcaccgatgatggccgtgtcttcctctggggctccttccgggacaataacggtgtgattggactgttggagcccatgaagaagagcatggtgcctgtgcaggtgcagctggatgtgcctgtggtaaaggtggcctcaggaaacgaccacttggtgatgctgacagctgatggtgacctctacaccttgggctgcggggaacagggccagctaggccgtgtgcctgagttatttgccaaccgtggtggccggcaaggcctcgaacgactcctggtccccaagtgtgtgatgctgaaatccaggggaagccggggccacgtgagattccaggatgccttttgtggtgcctatttcacctttgccatctcccatgagggccacgtgtacggcttcggcctctccaactaccatcagcttggaactccgggcacagaatcttgcttcataccccagaacctaacatccttcaagaattccaccaagtcctgggtgggcttctctggtggccagcaccatacagtctgcatggattcggaaggaaaagcatacagcctgggccgggctgagtatgggcggctgggccttggagagggtgctgaggagaagagcatacccaccctcatctccaggctgcctgctgtctcctcggtggcttgtggggcctctgtggggtatgctgtgaccaaggatggtcgtgttttcgcctggggcatgggcaccaactaccagctgggcacagggcaggatgaggacgcctggagccctgtggagatgatgggcaaacagctggagaaccgtgtggtcttatctgtgtccagcgggggccagcatacagtcttattagtcaaggacaaagaacagagctgatgaagcctctgagggcctggcttctgtcctgcacaacctccctcacagaacagggaagcagtgacagctgcagatggcagcgggcctctccccagccctgagcactgtgtcagttcctgccttttctcatcagcagaacagaatccttttcctcttttccttcctcctctttggaattttcctgggacctacagaataaagggggggatggacagggggttttcaaaaggaacatggctcactcagagctatatggttagacgtttctccccttttccctaccttccatggtcctggttggccctggctttgcctactagaaaaccaaaacttcccccctggggttttgtgcccactctctgagaagttggggctccatcaagccccattctagtcatgtgcccctttcctgtccctaacagtccacaggcaaacaaatggtacagtcataagagccatctgtcacggacccacgcccagaggaacgtgcagaaaaaagcagagctacatggctgtgggcaactataagccaaatatttggctcagaacaggtgtccatgggacaaaaaagaacgatcctccacttgaccaagaaaaaagtgattctcccagaagcacaaagcatactcttgcccctcaggtgttgcttgtgtacatcgtacccatccattcggcttcacctgcagccaacggcctggaatcgcaaagagacaccactctgggcagagcagagcagggtatggggtggggagagagggtggagggttttataaacaaacttaacagcaatattgaaaggaggtgggggattgagggagggacagagtgttggagggccagagactagtcctgagatggaaacagcaacttgtacagtggctgagaaaataggatatagttttgatttttttaattgtaaaatattttggagggagaacaaaatcttttaacattttgaataaatttagagttttataaaataggccacttgttttctacacattccctgctttttaagggagcacatattatgtgccaggcactgctgggaaagacagaataaactataaacctggtgttgaggctac

SEQ ID NO: 6 - DNA, 508 bp, human
cccagaacctaacatccttcaagaattccaccaagtcctgggtgggcttctctggtggccagcaccatacagtctgcatggattcggaaggaaaagcatacagcctgggccgggctgagtatgggcggctgggccttggagagggtgctgaggagaagagcatacccaccctcatctccaggctgcctgctgtctcctcggtggcttgtggggcctctgtggggtatgctgtgaccaaggatggtcgtgttttcgcctggggcatgggcaccaactaccagctgggcacagggcaggatgaggacgcctggagccctgtggagatgatgggcaaacagctggagaaccgtgtggtcttatctgtgtccagcgggggccagcatacagtcttattagtcaaggacaaagaacagagctgatgaagcctctgagggcctggcttctgtcctgcacaacctccctcacagaacagggaagcagtgacagctgcagatggcagcgggcctct

SEQ ID NO: 7 - DNA, 3350 bp, human
tagtcgcggggcaggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 8 - DNA, 497 bp, human
gtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccg

SEQ ID NO: 9 - DNA, 1460 bp, human
gacggcctggcatacccactgcccaccccagtgactgctcttctgcttcaggcctgctggcctcccagcactgcctgcccctccctgtcgggggacatcgcctccacaccggctggggaaggagcccaggggtggggctggtgggtggggctggtggttggggcagccagagaagtaagagggaagtgagaagccgggtggggcaggctggaaggaagacgaacctacgaagcagagatctgaagacagcatgtacacagccattccccagagtggctctccattcccaggctcagtgcaggatccaggcctgcatgtgtggcgggtggagaagctgaagccggtgcctgtggcgcaagagaaccagggcgtcttcttctcgggggactcctacctagtgctgcacaatggcccagaagaggtttcccatctgcacctgtggataggccagcagtcatcccgggatgagcagggggcctgtgccgtgctggctgtgcacctcaacacgctgctgggagagcggcctgtgcagcaccgcgaggtgcagggcaatgagtctgacctcttcatgagctacttcccacggggcctcaagtaccaggaaggtggtgtggagtcagcatttcacaagacctccacaggagccccagctgccatcaagaaactctaccaggtgaaggggaagaagaacatccgtgccaccgagcgggcactgaactgggacagcttcaacactggggactgcttcatcctggacctgggccagaacatcttcgcctggtgtggtggaaagtccaacatcctggaacgcaacaaggcgagggacctggccctggccatccgggacagtgagcgacagggcaaggcccaggtggagattgtcactgatggggaggagcctgctgagatgatccaggtcctgggccccaagcctgctctgaaggagggcaaccctgaggaagacctcacagctgacaaggcaaatgcccaggccgcagctctgtataaggtctctgatgccactggacagatgaacctgaccaaggtggctgactccagcccatttgcccttgaactgctgatatctgatgactgctttgtgctggacaacgggctctgtggcaagatctatatctggaaggggcgaaaagcgaatgagaaggagcggcaggcagccctgcaggtggccgagggcttcatctcgcgcatgcagtacgccccgaacactcaggtggagattctgcctcagggccatgagagtcccatcttcaagcaatttttcaaggactggaaatgagggtgggcgtcttcctgccccatgctcccctgccccccaccacctgcctgcttgcttctctggctgcctggtcagtgcagaggtgccccctgcagatgttcaataaaggagacaagtgctttcccagctcttttcctgcaccaccaaaaaaaaaaaaaaaaaa

SEQ ID NO: 10 - DNA, 299 bp, human
tggccatccgggacagtgagcgacagggcaaggcccaggtggagattgtcactgatggggaggagcctgctgagatgatccaggtcctgggccccaagcctgctctgaaggagggcaaccctgaggaagacctcacagctgacaaggcaaatgcccaggccgcagctctgtataaggtctctgatgccactggacagatgaacctgaccaaggtggctgactccagcccctttgcccttgaactgctgatatctgatgactgctttgtgctggacaacgggctctgtggcaagatctat

SEQ ID NO: 11 - DNA, 1767 bp, human
caggcgggcgggagggcgggcacggagaggcgggcgccgaggaggggcaggtagggctgggacgcaggggtaactggatcccccgacttcagcccaggccctggtctgaccaccctgggagcagggactttccacagtcagctggacgcacactcagcccagtaaaagaggggacccatcccgggagccccggggagggcacagctgcctcctcccgggctcccctgccacctggtgcctacctgccccctgctccctgccgggtccggtcctcaccccatcttcatctggccttgactctgcccttgaggggcctaggggtgcagccagcctgctccgagctcccctgcagatggaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgagggtgcgtctgggatccgaggtgcccaggctgggtggggagtctggggtccgtcctggactaggctgacccctgtgtcgttacccccagaaccagcctgtgaggatgcggatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaactgagggcctgtggaggccgagtctgtcctggaaccaggcttgcctgggacggctgagctgggcagctggaagtggctctggggtcctcacatggcgtcctgcccttgctccagcctgacaacagcctgagaaatccccccgtaacttattatcactttggggttcggcctgtgtcccccgaacgctctgcaccttctgacgcagcctgagaatgacctgccctggccccagccctactctgtgtaatagaataaaggcctgcgtgtgtctgtgttgagcgtgcgtctgtgtgtgcctgtgtgcgagtctgagtcagagatttggagatgtctctgtgtgtttgtgtgtatctgtgggtctccatcctccatgggggctcagccaggtgctgtgacaccccccttctgaatgaagccttctgacctgggctggcactgctgggggtgaggacacattgccccatgagacagtcccagaacacggcagctgctggctgtgacaatggtttcaccatccttagaccaagggatgggacctgatgacctgggaggactctcttagttcttaccttttgtggttctcaataaaacagaacttaaaaaattaaaaaaaaaaaaaaaaaaaaaaaa

SEQ ID NO: 12 - DNA, 251 bp, human
tgcctgtgtgcgagtctgagtcagagatttggagatgtctctgtgtgtttgtgtgtatctgtgggtctccatcctccatgggggctcagccaggtgctgtgacaccccccttctgaatgaagccttctgacctgggctggcactgctgggggtgaggacacattgccccatgagacagtcccagaacacggcagctgctggctgtgacaatggtttcaccatccttagaccaagggatgggacctgatgac

SEQ ID NO: 13 - DNA, 3474 bp, human
gagaactggacgatcgcctggcttagaagttttcctccctccccgaaccccgttttctcttccatttcttccggtcgcgtgtccccagcgcccacaggtggaaatcaaccgcccgcggggttgcggggcacaaagaggcagctagcggctccgctgaccccttcccgccggcctggacgaagtctgggctcgggagccgcgtgatgcatcccaaagaaggcgcagaacagcacgtgttctccccggtgcccggggctcccacaccaccgcccaatcgctgcggccgcctagtgctcgggccgcgcctgccggccgcggggactccgggcccgggtattcgcgccgccgccgcccgccatgcgcttccgctttggggtggtggtgccacccgccgtggccggcgcccggccggagctgctggtggtggggtcgcggcccgagctggggcgttgggagccgcgcggtgccgtccgcctgaggccggccggcaccgcggcgggcgacggggccctggccctgcaggagccgggcctgtggctcggggaggtggagctggcggccgaggaggcggcgcaggacggggcggagccgggccgcgtggacacgttctggtacaagttcctgaagcgggagccgggaggagagctctcctgggaaggcaatggacctcatcatgaccgttgctgtacttacaatgaaaacaacttggtggatggtgtgtattgtctcccaataggacactggattgaggccactgggcacaccaatgaaatgaagcacacaacagacttctattttaatattgcaggccaccaagccatgcattattcaagaattctaccaaatatctggctgggtagctgccctcgtcaggtggaacatgtaaccatcaaactgaagcatgaattggggattacagctgtaatgaatttccagactgaatgggatattgtacagaattcctcaggctgtaaccgctacccagagcccatgactccagacactatgattaaactatatagggaagaaggcttggcctacatctggatgccaacaccagatatgagcaccgaaggccgagtacagatgctgccccaggcggtgtgcctgctgcatgcgctgctggagaagggacacatcgtgtacgtgcactgcaacgctggggtgggccgctccaccgcggctgtctgcggctggctccagtatgtgatgggctggaatctgaggaaggtgcagtatttcctcatggccaagaggccggctgtctacattgacgaagaggccttggcccgggcacaagaagattttttccagaaatttgggaaggttcgttcttctgtgtgtagcctgtagctggtcagcctgcttctgccccctcctgatttccctaaggagcctgggatgatgttggtcaaatgacctagaaacaaggattctacctgaactgaaaggactgtgtgacctcccccaagccaaccactttcacctgggatgactttcgattatgctttgttttggggctgtatttttgaaatactctacaagaaagctgtggctcaacacatgagaagaagcacgaagcagttaggctgtacatcagacagaagggtaatgcgtgcagttcctgctgcctgcaggcagacgaggcctttgctttacagcactgtatgtgttgcacgatggatccgtgacagcactttcctgttgcactgaaactcttggccatgtagaggaaaagatatggagttatgtggatttcatcactagtatgtgtgcgtgagctggtcagttgccaaaggaggaaataaggttagaagcctgaaccgttacaaaagaagagctcactatggtcaaaaagtgatggctttcaggacttgttttttatcctgcctcacagttgttaaagtctgttccaaggcatcaccttccttctctacccaacaaccctgtgtaacaactaaagtagaattatctctcatttgttgttgtttttcctcaaaattaccaaacaaagcaaaaaatacccttgttttttatagttgagatgtcaagaagttaaattgaggcttaatgagcataggtagcttgtccaaggtctcatgaccagtcaagggcaagctggagttaataatctatatttatttgactcagcactgttttcatcacaacttgttttcccagcatcatgtagtgcatttagttttgtctttctcagggtatagtcaatatgcctgcaggagtttctatagcgagacatagaatagtattctgatcagttgccaaagaatctaggaaattagttgtattttgtgcaagctaatttaaaaacatgatgggctgttttaagaccagagtggaaattcatgagaggaactatactaccaaaagagcccaaatgaccaaatccatggataattgcttcacagccttggccatcctggctcagctctcaatttagtataatatgcagttcctgtgcctccagactatgcagctcatcaccctaggttctacaggaaatacagagatgaacaactttgccttcaaaaatgtgctgcctagaaacagacctgcattcaaccaactgtaatgcaggattggaccatgaatgatatgctagaatagaagaaagagaagtgtttttttaattgagagcctctatgtgcaaggtgatatataatcatatccagtttaatcttcacaatatccaatgaagaaggtctcattatctccatgataaagatggggaaactaaggtcagaagggttaactcaactgtctattgtcacatgatgaataaatagatgaagtgagatacaaagctgggtttgattcaaagcccttactttcctaattaaactatgatgcgtatttatttttctgcaccttcctttcttccacaaacacatattgatagatgcaagagactcttatttagaaggcgtgggggacaagaaggatacaaggtaagtttcagtggagctcagaggacggggagatagaactgtggcacttaggggagatgacatttgctttgggcagaggcagctagccaggacacatttccactataattttacaaagttaaatttataagctagcattaagtaaagtgaagtccagctcccttgctaaaaataactagaggtaataattggtattcaggtaactcatttacagtcataatgtgttgtgaaaatttaatcttaaaaattaaatttttaaactatgtgggtctgtgaatttctttaatgtctaagaaatccagcttcataatttccatgatacaaagatcttttttcaggtggatttttacctttgttccttttgctctgatagacaaaatcagtttaggactattaaagaatgttttggaataaactgtctttttcctcaatgaatgggatgtctaatgtatttcaaaatcacccaaaacttttggcaaataaaagcatttaaaaagacaaaaaaaaa

SEQ ID NO: 14 - DNA, 413 bp, human
gaaagctgtggctcaacacatgagaagaagcacgaagcagttaggctgtacatcagacagaagggtaatgcgtgcagttcctgctgcctgcaggcagacgaggcctttgctttacagcactgtatgtgttgcacgatggatccgtgacagcactttcctgttgcactgaaactcttggccatgtagaggaaaagatatggagttatgtggatttcatcactagtatgtgtgcgtgagctggtcagttgccaaaggaggaaataaggttagaagcctgaaccgttacaaaagaagagctcactatggtcaaaaagtgatggctttcaggacttgttttttatcctgcctcacagttgttaaagtctgttccaaggcatcaccttccttctctacccaacaaccctgtgtaacaa

SEQ ID NO: 15 - DNA, 25 bp, human
cattattcaaggccgagtacagatg

SEQ ID NO: 16 - DNA, 23 bp, human
cacgtacacgatgtgtcccttct

SEQ ID NO: 17 - DNA, 21 bp, human
caggcggtgtgcctgctgcat

SEQ ID NO: 18 - DNA, 22 bp, human
cctgaggactcacactggtgaa

SEQ ID NO: 19 - DNA, 17 bp, human
cagctcatccgatcgcg

SEQ ID NO: 20 - DNA, 27 bp, human
caagtgtacctgggaaggctgcgactg

SEQ ID NO: 21 - DNA, 24 bp, human
cgcagctctgtataaggtctctga

SEQ ID NO: 22 - DNA, 22 bp, human
gatatcagcagttcaagggcaa

SEQ ID NO: 23 - DNA, 26 bp, human
aacctgaccaaggtggctgactccag

SEQ ID NO: 24 - DNA, 21 bp, human
agatggacactgaggctgctg

SEQ ID NO: 25 - DNA, 22 bp, human
cttccgtctaagggtcaagctg

SEQ ID NO: 26 - DNA, 23 bp, human
cccaggatgtgacctacgcccag

SEQ ID NO: 27 - DNA, 18 bp, human
ctcccaccggacgccatc

SEQ ID NO: 28 - DNA, 21 bp, human
cctcgttctcgtagctcgcca

SEQ ID NO: 29 - DNA, 24 bp, human
cgggattctgatggtgccaacagt

SEQ ID NO: 30 - DNA, 23 bp, human
tttgtggtgcctatttcaccttt

SEQ ID NO: 31 - DNA, 21 bp, human
cggagttccaagctgatggta

SEQ ID NO: 32 - DNA, 22 bp, human
ccacgtgtacggcttcggcctc

SEQ ID NO: 33 - DNA, 22 bp, human
cctgtctcttgggaagcagttt

SEQ ID NO: 34 - DNA, 18 bp, human
gctcctgtgggctcaaag

SEQ ID NO: 35 - DNA, 25 bp, human
atcatgggcattgctggactgatgg

SEQ ID NO: 36 - DNA, 22 bp, human
aagccaccccacttctctctaa

SEQ ID NO: 37 - DNA, 22 bp, human
aatgctatcacctcccctgtgt

SEQ ID NO: 38 - DNA, 26 bp, human
agaatggcccagtcctctcccaagtc

SEQ ID NO: 39 - DNA, 19 bp, human
cctgcccactgtgcttcct

SEQ ID NO: 40 - DNA, 19 bp, human
ggttttcccgcttgcagat

SEQ ID NO: 41 - DNA, 15 bp, human
ctggcttcaccatcg

SEQ ID NO: 42 - DNA, 22 bp, human
cggaagaagaaacagctcatga

SEQ ID NO: 43 - DNA, 28 bp, human
cctctgtgtatttgtcaattttcttctc

SEQ ID NO: 44 - DNA, 17 bp, human
cggaaacaggccgagaa

(19) United States
(12) Patent Application Publication
(10) Pub. No.: US 2009/0192045 A1
(43) Pub. Date: Jul. 30, 2009

(54) MOLECULAR STAGING OF STAGE II AND III COLON CANCER AND PROGNOSIS

(76) Inventors: Yuqiu Jiang, San Diego, CA (US); Yi Zhang, San Diego, CA (US); Yixin Wang, Basking Ridge, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/355,906

(22) Filed: Jan. 19, 2009

Related U.S. Application Data
(60) Provisional application No. 61/022,539, filed on Jan. 22, 2008.

(51) Int. Cl. C40B 30/00; C12Q 1/68

(52) U.S. Cl. 506/7; 435/6

(57) ABSTRACT

Kits and articles include reagents for conducting a seven gene assay to stage colon cancer as Stage II or Stage III colon cancer.

### BACKGROUND OF THE INVENTION

Accurate staging of colon cancers not only contributes to disease prognosis prediction, but also to the clinical management and treatment selection of patients. The TNM system based on clinical and pathological features was introduced 1940s, and has gradually evolved and adopted in universal use since the 1980s. Quirke et al. (2007). In these guidelines, adequate lymph node evaluation is critical for appropriate staging of colon cancer. However, due to patient, surgeon, pathologist, and tumor related variables, 63% of colon cancer patients may not receive adequate lymph node evaluation. Baxter et al. (2005).

Genomic approaches have been successfully applied in identification of cancer classifications and sub-classifications, disease progression prediction, and treatment selection and treatment response prediction. Bhattacharjee et al. (2001); Khan et al. (2001); Sorlie et al. (2003); Agrawal et al. (2002); and Wang et al. (2005). The genetic and epigenetic information provides the opportunities to improve current cancer diagnostic and prognostic accuracy and could be complementary to clinical and pathological parameters. Using microarray analysis, a 23-gene prognostic signature for Stage II colon cancer patients has been developed. Wang et al. (2004). The signature has been further validated in independent samples from multiple clinical sites. Jiang et al. (2008). However, it is still believed that the prognostic value of gene signature may be enhanced through more accurate staging of the tumors.

### SUMMARY OF THE INVENTION

In one aspect of the invention, a diagnostic includes a 7-gene signature for determining whether colon cancer is in Stage II or Stage II.

In another aspect of the invention, a diagnostic includes reagents for detecting the expression of 7-genes used to distinguish between Stage II and Stage III colon cancer.

In yet another aspect of the invention, kits for distinguishing between Stage II and Stage III colon cancer and/or providing a prognosis of outcome include reagents for detecting the expression of 7-Marker genes and, optionally, a group of constitutively expressed genes.

### BRIEF DESCRIPTION OF THE DRAWINGS

FIG. 1. ROC and Kaplan-Meier survival analysis of the 7-gene signatures on 137 Stage II and III patients using Affymetrix microarray. A. The ROC curve of the 7-gene signature. B. Kaplan-Meier curve and log rank test of 137 frozen tumor samples using the 7-gene signature. The high and low risk groups differ significantly (P=0.007).

FIG. 2. ROC and Kaplan-Meier survival analysis of the 7-gene signatures on 123 FPE Stage II and III samples using RTQ-PCR. A. The ROC curve of the 7-gene signature. B. Kaplan-Meier curve and log rank test of 123 FPE samples using the 7-gene signature. The high and low risk groups differ significantly (P=0.0271).

FIG. 3. Kaplan-Meier survival analysis of the 7-gene signatures on 180 independent FPE Stage II colon cancer samples from 4 different clinic sites using RTQ-PCR.

### DETAILED DESCRIPTION OF THE INVENTION

One of the most important clinical factors for staging of Stage II and Stage III colon cancer is nodal involvement and the clinical guidelines recommend that at least 12 nodes need to be examined for proper staging. However, less than 40% of patients with colon cancer receive adequate lymph node evaluation. Baxter et al. (2005). A 23-gene prognostic signature to predict tumor recurrence in Stage II colon cancer has previously been referred to, in for example, US Patent Publication 20060063157 which is incorporated in its entirety herein by reference. Subsequently, this signature was validated in an independent patient group of 123 Stage II colon cancers using fresh frozen tumor specimens and a group of 110 Stage II patients using formalin-fixed paraffin embedded samples. Jiang et al. (2008). The present invention is directed to more accurate staging.

A Biomarker is any indicia of an indicated Marker nucleic acid/protein. Nucleic acids can be any known in the art including, without limitation, nuclear, mitochondrial (homeoplasmy, heteroplasmy), viral, bacterial, fungal, mycoplasmal, etc. The indicia can be direct or indirect and measure over- or under-expression of the gene given the physiologic parameters and in comparison to an internal control, placebo, normal tissue or another carcinoma. Biomarkers include, without limitation, nucleic acids and proteins (both over and under-expression and direct and indirect). Using nucleic acids as Biomarkers can include any method known in the art including, without limitation, measuring DNA amplification, deletion, insertion, duplication, RNA, microRNA (miRNA), loss of heterozygosity (LOH), single nucleotide polymorphisms (SNPs, Brookes (1999)), copy number polymorphisms (CNPs) either directly or upon genome amplification, microsatellite DNA, epigenetic changes such as DNA hypo- or hyper-methylation and FISH. Using proteins as Biomarkers includes any method known in the art including, without limitation, measuring amount, activity, modifications such as glycosylation, phosphorylation, ADP-ribosylation, ubiquitination, etc., or immunohistochemistry (IHC) and turnover. Other Biomarkers include imaging, molecular profiling, cell count and apoptosis Markers.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. No. 5,445,934; U.S. Pat. No. 5,532,128; U.S. Pat. No. 5,556,752; U.S. Pat. No. 5,242,974; U.S. Pat. No. 5,384,261; U.S. Pat. No. 5,405,783; U.S. Pat. No. 5,412,087; U.S. Pat. No. 5,424,186; U.S. Pat. No. 5,429,807; U.S. Pat. No. 5,436,327; U.S. Pat. No. 5,472,672; U.S. Pat. No. 5,527,681; U.S. Pat. No. 5,529,756; U.S. Pat. No. 5,545,531; U.S. Pat. No. 5,554,501; U.S. Pat. No. 5,561,071; U.S. Pat. No. 5,571,639; U.S. Pat. No. 5,593,839; U.S. Pat. No. 5,599,695; U.S. Pat. No. 5,624,711; U.S. Pat. No. 5,658,734; and U.S. Pat. No. 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. No. 6,271,002; U.S. Pat. No. 6,218,122; U.S. Pat. No. 6,218,114; and U.S. Pat. No. 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All Markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “Genespring” (Silicon Genetics, Inc.) and “Discovery” and “Infer” (Partek, Inc.)

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Methods of isolating nucleic acid and protein are well known in the art. See e.g. U.S. Pat. No. 6,992,182 incorporated by reference herein in its entirety and the discussion of RNA isolation at the Ambion website on the World Wide Web of the Internet, and US 20070054287.

DNA analysis can be any known in the art including, without limitation, methylation, de-methylation, karyotyping, ploidy (aneuploidy, polyploidy), DNA integrity (assessed through gels or spectrophotometry), translocations, mutations, gene fusions, activation—de-activation, single nucleotide polymorphisms (SNPs), copy number or whole genome amplification to detect genetic makeup. RNA analysis includes any known in the art including, without limitation, q-RT-PCR, miRNA or post-transcription modifications. Protein analysis includes any known in the art including, without limitation, antibody detection, post-translation modifications or turnover. The proteins can be cell surface markers, preferably epithelial, endothelial, viral or cell type. The Biomarker can be related to viral/bacterial infection, insult or antigen expression.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The following examples are provided to illustrate but not limit the invention.

### Example 1

### Materials and Methods

### Patient Samples

Frozen tumor specimens from 78 coded Stage II and 59 Stage III colon cancer patients were obtained. Archived primary tumor samples were collected at the time of surgery. The histopathology of each specimen was reviewed on the H&E stained tissue section to confirm diagnosis and tumor content. Tumor content was estimated in percentage by counting nuclei of epithelial tumor cells. Patient eligibility criteria include: colon primary Stage II and III adenocarcinoma, primary treatment is surgery only without adjuvant or neo-adjuvant therapy, at least 70% of tumor cells in the tissue sample, and at least 3 years of follow-up except for patients who developed distant relapse before that time. Post-surgery patient surveillance was carried out according to general practice for colon cancer patients including physical exam, blood counts, liver function tests, serum CEA, and colonoscopy for the patients. Selected patients had abdominal CT scan and chest X-ray. If cancer recurrence was suspected, the patient underwent diagnostic work-up including colonoscopy, chest/abdominal/pelvic CT and MRI for selected patients. Diagnostic biopsy to confirm metastatic lesion was performed in all patients where feasible. Time to recurrence or disease-free time was defined as the time period from the date of surgery to confirmed tumor relapse date for relapsed patients and from the date of surgery to the date of last follow-up for disease-free patients.

FPE tumor specimens from 85 Stage II and 38 Stage III colon cancer patients were also obtained. There were also 180 Stage II colon cancer FPE specimens acquired separately. The histopathology of each specimen was reviewed to confirm diagnosis and tumor content. Patient eligibility criteria and follow-up procedures were the same as for the selection of the frozen samples.

### Microarray Analysis

All frozen tumor tissues were processed for RNA isolation. Baxter et al. (2005). Biotinylated targets were prepared using published methods (Affymetrix, Santa Clara, Calif.) and hybridized to Affymetrix U133a GeneChips (Affymetrix, Santa Clara, Calif.). Arrays were scanned using the standard Affymetrix protocol. Each probe set was considered a separate gene. Expression values for each gene were calculated using Affymetrix GeneChip® analysis software MAS 5.0 and according to the analysis method described previously. Wang et al. (2004).

RNA Isolation from FPE Samples

The FPE samples were either formalin-fixed (n=45) or Hollandes-fixed (n=65) FPE tissues. RNA isolation from FPE tissue samples was carried out according to a modified protocol using High Pure RNA Paraffin Kit (Roche Applied Sciences, Indianapolis, Ind.). FPE tissue blocks were sectioned depending on the size of the blocks (6-8 mm=6×10 μm, ≧8 mm=3×10 μm). Sections were de-paraffinized as described in the manufacturer's manual. The tissue pellet was dried in oven at 55° C. for 10 minutes and resuspended in 100 μL of tissue lysis buffer, 16 μL 10% SDS and 80 μL Proteinase K. The sample was vortexed and incubated in a thermomixer set at 400 rpm for 3 hours at 55° C. Subsequent steps of sample processing were performed according the Kit manual. The RNA sample was quantified by OD 260/280 readings using spectrophotometer and diluted to a final concentration of 50 ng/uL. The isolated RNA samples were stored in RNase-free water at −80° C. until use.

### RTQ-PCR Analysis

The gene signature and the housekeeping control genes were evaluated using a one-step multiplex RTQ-PCR assay with the RNA samples isolated from FPE tissues. In order to minimize the variability of RTQ-PCR reaction, three housekeeping control genes including β-actin, HMBS, and RPL13A, were used to normalize the input quantity of RNA. To prevent any contaminating DNA in the samples from amplification, PCR primers or probes for RTQ-PCR assay were designed to span an intron so that the assay would not amplify any residual genomic DNA. One hundred nanograms of total RNA were used for the one-step RTQ-PCR reaction. The reverse transcription was carried out using 40× Multiscribe and RNase inhibitor mix contained in the TaqMan® one-step PCR Master Mix reagents kit (Applied Biosystems, Fresno, Calif.). The cDNA was then subjected to the 2× Master Mix without uracil-N-glycosylase (UNG). PCR amplification was performed on the ABI 7900HT sequence detection system (Applied Biosystems, Frenso, Calif.) using the 384-well block format with 10 μL reaction volume. The concentrations of the primers and the probes were 4 and 2.5 μmol/L, respectively. The reaction mixture was incubated at 48° C. for 30 minutes for the reverse transcription, followed by an Amplitaq® activation step at 95° C. for 10 minutes and then 40 cycles of 95° C. for 15 seconds for denaturing and of 60° C. for 1 minute for annealing and extension. A standard curve was generated from a range of 100 pg to 100 ng of the starting materials, and when the R² value was >0.99, the cycle threshold (Ct) values were accepted. In addition, all primers and probes were optimized towards the same amplification efficiency according to the manufacturer's protocol. Sequences of the primers and probes for the 7 genes and the 3 housekeeping control genes were as follows, each written in the 5′ to 3′ direction:

| | |
|---|---|
| EP2MA | CATTATTCAAGGCCGAGTACAGATG; |
| forward, | |
| EP2MA | CACGTACACGATGTGTCCCTTCT; |
| reverse, | |
| EP2MA | FAM-CAGGCGGTGTGCCTGCTGCAT-BHQ. |
| probe, | |
| KLF5 | CCTGAGGACTCACACTGGTGAA; |
| forward, | |
| KLF5 | CAGCTCATCCGATCGCG; |
| reverse, | |
| KLF5 | FAM-CAAGTGTACCTGGGAAGGCTGCGACTG-BHQ. |
| probe, | |
| CAPG | CGCAGCTCTGTATAAGGTCTCTGA; |
| forward, | |
| CAPG | GATATCAGCAGTTCAAGGGCAA; |
| reverse, | |
| CAPG | FAM-AACCTGACCAAGGTGGCTGACTCCAG-BHQ. |
| probe, | |
| LILRB3 | AGATGGACACTGAGGCTGCTG; |
| forward, | |
| LILRB3 | CTTCCGTCTAAGGGTCAAGCTG; |
| reverse, | |
| LILRB3 | FAM-CCCAGGATGTGACCTACGCCCAG-BHQ. |
| probe, | |
| LAT | CTCCCACCGGACGCCATC; |
| forward, | |
| LAT | CCTCGTTCTCGTAGCTCGCCA; |
| reverse, | |
| LAT probe, | FAM-CGGGATTCTGATGGTGCCAACAGT-BHQ-1-TT. |
| CHC1 | TTTGTGGTGCCTATTTCACCTTT; |
| forward, | |
| CHC1 | CGGAGTTCCAAGCTGATGGTA; |
| reverse, | |
| CHC1 probe, | FAM-CCACGTGTACGGCTTCGGCCTC-BHQ. |
| YWHAH | CCTGTCTCTTGGGAAGCAGTTT; |
| forward, | |
| YWHAH | GCTCCTGTGGGCTCAAAG; |
| reverse, | |
| YWHAH | FAM-ATCATGGGCATTGCTGGACTGATGG-BHQ. |
| probe, | |
| β-actin | AAGCCACCCCACTTCTCTCTAA; |
| forward, | |
| β-actin | AATGCTATCACCTCCCCTGTGT; |
| reverse, | |
| β-actin | FAM-AGAATGGCCCAGTCCTCTCCCAAGTC-BHQ. |
| probe, | |
| HMBS | CCTGCCCACTGTGCTTCCT; |
| forward, | |
| HMBS | GGTTTTCCCGCTTGCAGAT; |
| reverse, | |
| HMBS probe, | FAM-CTGGCTTCACCATCG-BHQ. |
| RPL13A | CGGAAGAAGAAACAGCTCATGA; |
| forward, | |
| RPL13A | CCTCTGTGTATTTGTCAATTTTCTTCTC; |
| reverse, | |
| RPL13A | FAM-CGGAAACAGGCCGAGAA-BHQ. |
| probe, | |

For each sample ΔCt=Ct (target gene)−Ct (average of four control genes) was calculated. ΔCt normalization has been widely used in clinical RTQ-PCR assay.

### Statistical Methods

t tests were used to compare the discrimination of each gene between the Stage II colon cancer patients and the Stage III colon cancer patients. Logistic regression was used on the CCF patients as the training set to build a model to assess the likelihood of being Stage III. The probabilities from the logistic model for each patients being Stage III were used to generate the Receiver's Operating Characteristic (ROC) curves. The threshold of the probabilities was chosen from the ROC curve to produce at least 90% specificity (90% of Stage II patients correctly identified). The model built from the training set was then used to compute the probabilities of being Stage III for patients of one of the testing sets. Kaplan Meier survival curves (Kaplan et al. (1958) and the hazard ratios calculated from Cox proportional hazards regression were used to assess the difference in recurrence free survival between the predicted Stage II and the predicted Stage III patients. All statistical analyses were performed using S-Plus® 6-1 software (Insightful, Fairfax Station, Va.).

### Results

### Patient and Tumor Characteristics

Clinical and pathological features of the patients and their tumors are summarized in Table 1 and Table 2.

TABLE 1
Patient and tumor characteristics Cleveland Clinic
Foundation Fresh Frozen and FPE samples

| | Cleveland Clinic Fresh Frozen | | | | Cleveland Clinic FFPE | | | |
|---|---|---|---|---|---|---|---|---|
| | Stage II | | Stage III | | Stage II | | Stage III | |
| Factor | # | % | # | % | # | % | # | % |
| Average | 70 | | 67 | | 69 | | 65 | |
| age (yr) | | | | | | | | |
| Sex | | | | | | | | |
| Male | 40 | 51 | 32 | 54 | 46 | 54 | 20 | 53 |
| Female | 38 | 49 | 27 | 46 | 39 | 46 | 18 | 47 |
| T Stage | | | | | | | | |
| T1 | 0 | 0 | 10 | 17 | 0 | 0 | 4 | 10 |
| T2 | 68 | 87 | 35 | 59 | 75 | 88 | 25 | 66 |
| T3 | 10 | 13 | 14 | 24 | 10 | 12 | 9 | 24 |
| Grade | | | | | | | | |
| Good | 7 | 9 | 3 | 5 | 9 | 10 | 1 | 3 |
| Moderate | 57 | 73 | 40 | 68 | 61 | 72 | 26 | 68 |
| Poor | 14 | 18 | 16 | 27 | 15 | 18 | 11 | 29 |
| Metastasis | | | | | | | | |
| Yes | 7 | 9 | 22 | 37 | 14 | 16 | 14 | 37 |
| No | 71 | 91 | 37 | 63 | 71 | 84 | 24 | 63 |

| | | | | |
|---|---|---|---|---|
| Median # LN | 28 (2-165) | 31 (2-333) | 29 (2-165) | 38 (8-333) |
| examined | | | | |

TABLE 2
Patient and tumor characteristics of
180 validation samples (FPE tissues)

| | Mayo | | | | San Diego Sharp | | | |
|---|---|---|---|---|---|---|---|---|
| | Clinic | | Oridis | | Hospital | | Proteogenex | |
| Factor | # | % | # | % | # | % | # | % |
| Average | 73 | | 68 | | 80 | | 64 | |
| age (yr) | | | | | | | | |
| Sex | | | | | | | | |
| Male | 26 | 38 | 28 | 55 | 12 | 48 | 14 | 29 |
| Female | 43 | 62 | 23 | 45 | 13 | 52 | 35 | 71 |
| T Stage | | | | | | | | |
| T2 | 0 | 0 | 0 | 0 | 0 | 0 | 2 | 4 |
| T3 | 66 | 96 | 43 | 84 | 22 | 88 | 40 | 82 |
| T4 | 3 | 4 | 8 | 16 | 3 | 12 | 7 | 14 |
| Grade | | | | | | | | |
| Good | 0 | 0 | 1 | 2 | 1 | 4 | 5 | 10 |
| Moderate | 34 | 49 | 36 | 71 | 23 | 92 | 8 | 16 |
| Poor | 28 | 41 | 14 | 27 | 1 | 4 | 3 | 6 |
| Unknown | 7 | 10 | 0 | 0 | 0 | 0 | 33 | 68 |
| Metastasis | | | | | | | | |
| Yes | 14 | 20 | 18 | 35 | 4 | 16 | 15 | 31 |
| No | 55 | 80 | 33 | 65 | 21 | 84 | 34 | 69 |

| | | | | |
|---|---|---|---|---|
| Median # LN | 13 (3-32) | 19 (6-72) | 29 (2-165) | 10 (2-38) |
| examined | | | | |

All patients had information on age, gender, TNM stage, number of lymph nodes examined, grade, and tumor location. All the patients had sporadic colon cancer. Rectal cancer patient was excluded from the study. TNM staging was performed according to AJCC 6^{th} edition guidelines. Histological grade or differentiation status was also reported by each clinical site. The number of lymph nodes examined varied among the sites because the samples came from the archived collections at different time periods. The patients were treated by surgery only and none of the patients received neo-adjuvant or adjuvant treatment. A minimum of 3 years of follow-up data was available for all the patients in the study with the exception of those with relapse or death in less than 3 years. The statistical analysis suggested that the tumor characteristics did not differ significantly between the relapse and the non-relapse patients. Analysis of the Gene Signature in the Fresh Frozen Samples.

In the patient sample group of an earlier study (Wang et al. (2004)), two subgroups were detected of tumors representing well- and poorly-differentiated tumors, respectively. Cadherin 17 gene expression was used to stratify the Stage II tumors into the two subgroups and the prognostic gene signature was designed to include classifiers for subgroup I (7 genes) and subgroup II (15 genes). In the present study, it was found that subgroup II (undetectable Cadherin 17) only accounted for 1 of the 78 Stage II tumors (1.3%) and 1 of the 59 Stage III tumors (1.7%). Therefore, an improved gene signature was formulated that includes only the 7 genes for subgroup I in the algorithm for current studies. The 7 genes are listed in Table 3 as follows with GenBank ID and Affymetrix U133a chip ID.

TABLE 3

| Gene | Seq ID No |
|---|---|
| LILRB3 | 1 |
| YWHAH | 3 |
| RCC1 | 5 |
| KLF5 | 7 |
| CAPG | 9 |
| LAT | 11 |
| EPM2A | 13 |

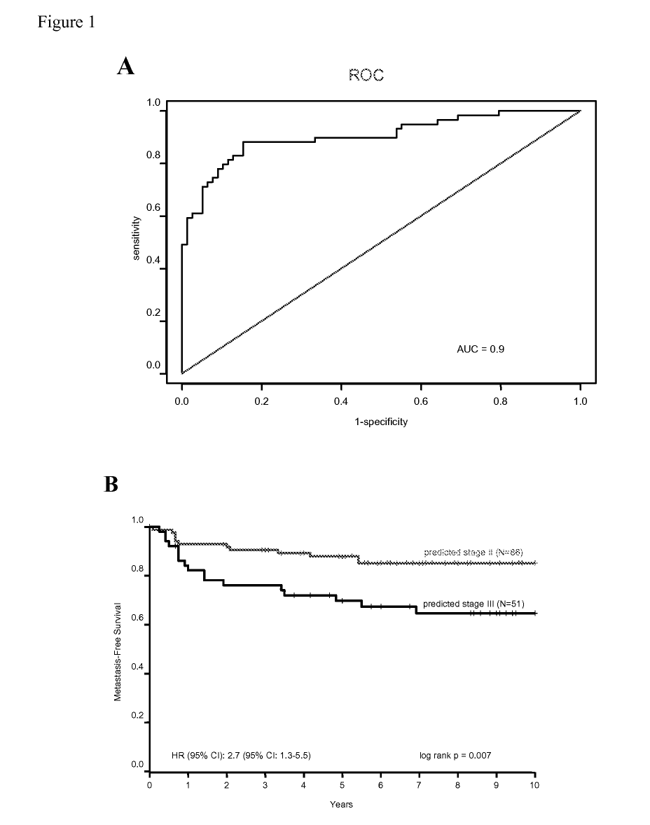

To assess the staging property of the 7-gene signature, we first used t test to compare the discrimination power of the 7 genes for differentiating the clinically defined Stage II and III patients. Then logistic regression was applied to the 137 samples to build a model to evaluate the likelihood of each patient being Stage III or Stage II. The parameter that was used to assess the performance of the 7-gene signature as a stage predictor was the area under the curve (AUC) of Receiver's Operating Characteristic (ROC) analysis. As shown in FIG. 1A, the signature gave an AUC value of 0.9.

The Kaplan-Meier analysis produced survival curves for the predicted Stage II and III patients (FIG. 1B). Clearly, the predicted Stage II and III patients segregated into two distinct clusters of patients with good prognosis (predicted Stage II patients) and poor prognosis (predicted Stage III patients). In the univariate Cox proportional hazards regression model, the estimated relative risk for tumor recurrence was 2.7 (95% CI, 1.3-5.5, P=0.007). Analysis of the Gene Signature in the FPE Samples

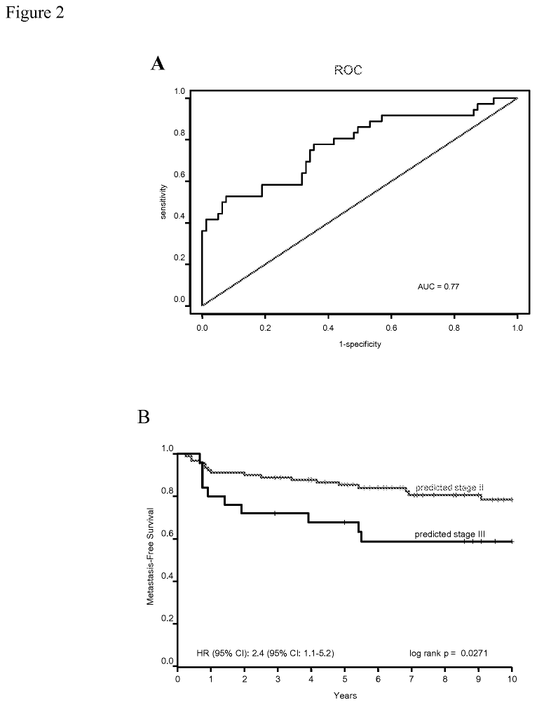

In order to demonstrate the staging value of the 7-gene signature in clinically relevant samples, RTQ-PCR assay was developed and performed first on 123 FPE samples from Stage II and III colon tumors. Since the RTQ-PCR assay is entirely different from the microarray analysis, in terms of the sample type and assay platform, the Stage discrimination power of the 7 genes were reevaluated by t test. A model to evaluate the likelihood of each patient being Stage III or Stage II was built again using logistic regression on these 123-patient RTQ-PCR dataset. First, the ROC curve was evaluated (FIG. 2A). The 7-gene predictor gave an AUC value of 0.77.

The Kaplan-Meier analysis and the log rank test both showed a significant difference in the time to recurrence between the group with predicted Stage III cancer and the group with predicted Stage II cancer (HR 2.4, 95% CI 1.1-5.2; P=0.02) (FIG. 2B).

Evaluation of an Independent Test Set from 4 Different Clinical Sites

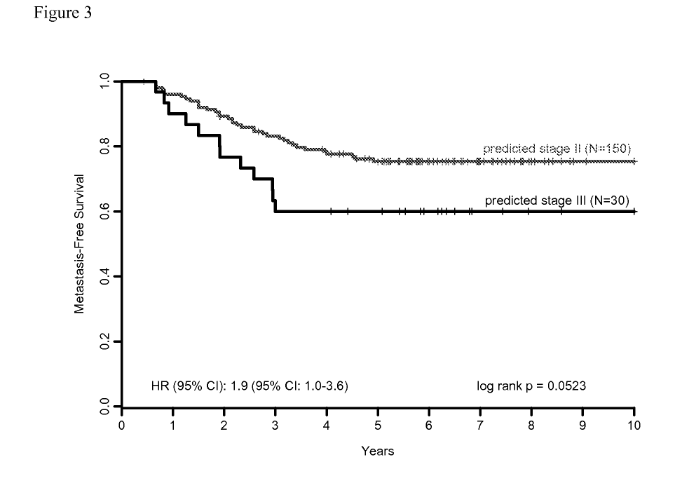

The 7-gene signature has been tested on clinically defined Stage II and III colon cancers and it was demonstrated that the signature has the ability to differentiate these two classes with fresh frozen specimen on microarray platform and with FPE specimen on RTQ-PCR platform. To test whether the predefined 7-gene signature would be able to differentiate the good prognosis patients from the poor prognosis patients for the clinically defined Stage II colon cancers, 180 test-set samples were used to assess the 7-gene utility. By applying the predefined model and algorithm obtained from the 123 Stage II and III sample set, 150 of the 180 clinical Stage II patients were classified as predicted Stage II cancers and 30 clinical Stage II patients were classified as predicted Stage III cancers. The Kaplan-Meier analysis and the log rank test both showed a significant difference in the time to recurrence between the group with predicted Stage III cancer and the group with predicted Stage II cancer (HR 2.0, 95% CI 1.0-3.6; P=0.05), as shown in FIG. 3.

### REFERENCES CITED

20030194734
20070054287
U.S. Pat. No. 5,424,186
U.S. Pat. No. 5,529,756
U.S. Pat. No. 5,532,128
U.S. Pat. No. 5,545,531
U.S. Pat. No. 5,556,752
U.S. Pat. No. 5,561,071
Agrawal et al. (2002) Osteopontin identified as lead marker of colon cancer progression, using pooled sample expression profiling J Natl Cancer Inst 94:513-521
Baxter et al. (2005) Lymph node evaluation in colorectal cancer patients: a population-based study J Natl Cancer Inst 97:219-25
Benson et al. (2004) American Society of Clinical Oncology recommendations on adjuvant chemotherapy for stage II colon cancer J Clin Oncol 22:3408-19
Bhattacharjee et al. (2001) Classification of human lung carcinomas by mRNA expression profiling reveals distinct adenocarcinoma subclasses Proc Natl Acad Sci USA 98:13790-13795
Brookes (1999) The essence of SNPs Gene 234:177-186
Chang et al. (2007) Lymph node evaluation and survival after curative resection of colon cancer: systematic review J Natl Cancer Inst 99:433-41
Eschrich et al. (2005) Molecular staging for survival prediction of colorectal cancer patients J Clin Oncol 23:3526-35
Jiang et al. (2008) Molecular signature classifies Stage II and III colon cancer and predicts tumor recurrence Submitted to J Mol Diag
Johnson et al. (2002) Adequacy of nodal harvest in colorectal cancer: a consecutive cohort study J Gastrointest Surg 6:883-88
Kaplan et al. (1958) Non-parametric estimation of incomplete observations J Am Stat Assoc 53:457-481
Khan et al. (2001) Classification and diagnostic prediction of cancers using gene expression profiling and artificial neural networks Nat Med 7:673-679
Liefers et al. (1998) Micrometastases and survival in stage II colorectal cancer N Engl J Med 339:223-8
Markowitz (1952) Portfolio Selection
Moertel et al. (1005) Fluorouracil plus levamisole as effective adjuvant therapy after resection of stage III colon carcinoma: A final report Ann Intern Med 122:321-326
Quirke et al. (2007) The future of the TNM staging system in colorectal cancer: time for a debate? Lancet Oncol 8:651-7
Ramaswamy et al. (2003) A molecular signature of metastasis in primary solid tumors Nat Genet 33:49-54
Saltz et al. (1997) Adjuvant treatment of colorectal cancer Annu Rev Med 48:191-202
Sorlie et al. (2003) Repeated observation of breast tumor subtypes in independent gene expression data sets Proc Natl Acad Sci USA 100:8418-8423
Tusher et al. (2001) Significance analysis of microarrays applied to the ionizing radiation response Proc Natl Acad Sci USA 98:5116-5121
Van de Vijver et al. (2002) A gene-expression signature as a predictor of survival in breast cancer N Engl J Med 347:1999-2009
Van't Veer et al. (2002) Gene expression profiling predicts clinical outcome of breast cancer Nature 415:530-6
Wang et al. (2004) Gene expression profiles and molecular markers to predict recurrence of Dukes' B colon cancer J Clin Oncol 22:1564-71
Wang et al. (2005) Gene-expression profiles to predict distant metastasis of lymph-node-negative primary breast cancer Lancet 365:671-9
Wang et al. (2006) Epm2a suppresses tumor growth in an immunocompromised host by inhibiting Wnt signaling Cancer Cell 10:179-90
Wolmark et al. (1999) Clinical trial to assess the relative efficacy of fluorouracil and leucovorin, fluorouracil and levamisole, and fluorouracil, leucovorin, and levamisole in patients with Dukes' B and C carcinoma of the colon: Results from National Surgical Adjuvant Breast and Bowel Project C-04 J Clin Oncol 17:3553-3559 www.ambion.com/techlib/basics/rnaisol/index.html
Yu et al. (2002) The 41/ezrin/radixin/moesin domain of the DAL-1/Protein 41B tumour suppressor interacts with 14-3-3 proteins Biochem J 365(Pt 3):783-9
Ziemer et al. (2001) Identification of a mouse homolog of the human BTEB2 transcription factor as a beta-catenin-independent Wnt-1-responsive gene Mol Cell Biol 21:562-74

We claim:

1. A method of staging colorectal cancer status comprising identifying differential modulation in a combination of genes consisting essentially of Seq ID NO1, Seq ID NO 3, Seq ID NO 5, Seq ID NO 7, Seq ID NO9, Seq ID NO11, and Seq ID NO 13.

2. The method of claim 1 wherein Stage II and Stage III colorectal cancer are distinguished.

3. The method of claim 2 wherein the comparison of expression patterns is conducted with pattern recognition methods.

4. The method of claim 3 wherein the pattern recognition methods include the use of a Cox proportional hazards analysis.

5. The method of claim 1 conducted on primary tumor sample.

6. The method of claim 1 wherein if the gene expression pattern of a sample is that of the patter of the Cox proportional hazard analysis indicating Stage II then the colorectal cancer is Stage II colorectal cancer and if it is not then it is Stage III colorectal cancer.

7. A kit for staging colorectal cancer patient comprising materials for detecting isolated nucleic acid sequences, their compliments, or portions thereof of a combination of genes that includes Seq ID NO1, Seq ID NO 3, Seq ID NO 5, Seq ID NO 7, Seq ID NO9, Seq ID NO11, and Seq ID NO 13.

8. The kit of claim 7 wherein the only combination of genes is Seq ID NO1, Seq ID NO 3, Seq ID NO 5, Seq ID NO 7, Seq ID NO9, Seq ID NO11, and Seq ID NO 13 and housekeeping or control genes.

9. The kit of claim 8 further comprising reagents for conducting a microarray analysis.

10. The kit of claim 9 further comprising a medium through which said nucleic acid sequences, their compliments, or portions thereof are assayed.

11. Articles for assessing colorectal cancer status comprising materials for identifying nucleic acid sequences, their complements, or portions thereof of a combination of genes that includes Seq ID NO 1, Seq ID NO 3, Seq ID NO 5, Seq ID NO 7, Seq ID NO9, Seq ID NO11, and Seq ID NO 13.

12. The article of claim 11 wherein the only combination of genes is Seq ID NO1, Seq ID NO 3, Seq ID NO 5, Seq ID NO 7, Seq ID NO9, Seq ID NO11, and Seq ID NO 13 and housekeeping or control genes.

13. The article of claim 12 further comprising reagents for conducting a microarray analysis.

14. The article of claim 13 further comprising a medium through which said nucleic acid sequences, their compliments, or portions thereof are assayed.

15. The article of claim 11 comprising reagents for conducting a PCR reaction wherein said reagents include probes and primers for detecting genes consisting essentially of Seq ID NO1, Seq ID NO 3, Seq ID NO 5, Seq ID NO 7, Seq ID NO9, Seq ID NO11, and Seq ID NO 13 and housekeeping or control genes.

16. The article of claim 15 further comprising instructions for analyzing the results of the use of the kit to stage colorectal cancer.

17. The article of claim 16 wherein the instructions are computer instructions.

18. The article of claim 17 wherein the computer instructions are contained on a magnetic or optical medium.
